(19) United States
(12) Patent Application Publication
(10) Pub. No.: US 2024/0082140 A1
(43) Pub. Date: Mar. 14, 2024

(54) NATURAL OIL-BASED PETROLATUM AND METHOD OF MAKING SAME

(71) Applicant: CARGILL, INCORPORATED, Wayzata, MN (US)

(72) Inventors: Emmanuel Paul Jos Marie EVERAERT, Poissy (FR); Todd L. KURTH, Maple Grove, MN (US); Timothy Alan MURPHY, Albertville, MN (US); Yijun ZHOU, Maple Grove, MN (US)

(73) Assignee: CARGILL, INCORPORATED, Wayzata, MN (US)

(21) Appl. No.: 18/260,109

(22) PCT Filed: Jan. 5, 2022
(86) PCT No.: PCT/US2022/070041

§ 371 Date: Jun. 30, 2023

Related U.S. Application Data
(60) Provisional application No. 63/156,563, filed on Mar. 4, 2021.
(60) Provisional application No. 63/134,015, filed on Jan. 5, 2021.

(51) Int. Cl. A61K 8/85

(52) U.S. Cl. CPC A61K 8/85; A61K 2800/10; A61K 2800/805

(57) ABSTRACT

The disclosure relates to natural oil-based petrolatum compositions and a method of making the same. The natural oil-based petrolatum composition includes the esterification product of a pre-esterification mixture of: about 15 wt % to about 25 wt % a fatty acid dimer, about 15 wt % to about 25 wt % glycerin, and about 55 wt % to about 70 wt % hydrogenated natural oil, wherein the natural oil-based petrolatum product has an acid value of less than 5.0. Natural oil-based petrolatum compositions can be used in personal care products.

### CROSS-REFERENCE TO RELATED APPLICATIONS

This application claims the benefit of U.S. Provisional Application No. 63/134,015, filed Jan. 5, 2021, and U.S. Provisional Application No. 63/156,563, filed Mar. 4, 2021, each of which is incorporated by reference herein in its entirety.

### TECHNICAL FIELD

This application relates to natural oil-based petrolatum compositions and methods of making the same.

### BACKGROUND

Petrolatum is a byproduct of petroleum refining. With a melting point close to body temperature, petrolatum softens upon application and forms a water-repellant film around the applied area, creating an effective barrier against the evaporation of the skin's natural moisture and foreign particles or microorganisms that may cause infection. Petrolatum is odorless and colorless, and it has an inherently long shelf life. It is not a single entity but rather comprised of a complex mixture of organic compounds with a wide diversity of molecular weights. This diversity of components allows petrolatum to have unique rheological properties over a wide variety of temperatures. For example, petrolatum does not have a distinct melting point like one traditionally thinks about in organic compounds. These properties make petrolatum a useful and popular ingredient in skincare products and cosmetics. It is often used as an ingredient in a wide variety of personal care products such as skin creams, lotions, hair care products and cosmetics. A primary benefit is petrolatum's occlusive properties where it can create a barrier to protect or preserve hydration of the skin. Therefore, it is commonly used to protect skin, hair, and lips or to aid in the healing of damaged skin or lips. It is most commonly known by the brand name Vaseline®.

When properly refined, petrolatum has no known health concerns. However, with an incomplete refining history, petrolatum could potentially be contaminated with polycyclic aromatic hydrocarbons, or PAHs. PAHs are byproducts of organic material combustion, commonly stored in fats upon exposure due to its lipophilic properties.

There have been numerous efforts to develop a bio-based alternative to petrolatum. Most of these efforts relate to creating blends of higher melting waxes, hydrogenated oils, or other natural oils. Through blending it may be possible to create a product with a similar feel to petrolatum, however these products suffer from a common disadvantage. Because they are simple blends, the rheology of the material does not match petrolatum as they are heated. The lower melting components melt first and while higher melting components remain intact until the temperature reaches a higher point. Put another way, these substitute products do not have a smooth melting curve, or smooth change in rheology over a range of temperatures. Rather they have duel or multiple phased melting profiles so they do not mimic the performance of petrolatum over a variety of temperatures. In addition, these blend can have a much higher Iodine Value (IV) representing the presence of a significantly high degree of unsaturation in the oils. This degree of unsaturation is undesirable because it contributes to significantly lower oxidative stability over time. The lower IV of the natural based petrolatum disclosed herein lead to improved oxidative stability and correspondingly improved shelf life and quality.

Accordingly, it would be advantageous to have improved natural based materials that more closely mimic the texture, viscosity, stability, and melting profiles of petrolatum. It would be environmentally and economically desirable if such materials were biodegradable and derived from renewable raw materials, such as natural oils.

### SUMMARY

In contrast to a simple blend of a few ingredients, the compositions disclosed herein more closely mimic petroleum based petrolatum by containing a complex mixture of components with differing molecular weights and rheological properties. Creating such a product by blending would be exhaustively time consuming and costly.

The present disclosure provides a natural oil-based petrolatum composition comprising the esterification product of a fatty acid dimer, a hydrogenated natural oil, and glycerin, wherein the natural oil based petrolatum composition has an acid value (AV) of less than about 5.0.

The present disclosure provides a natural oil-based petrolatum composition that includes an esterification product of a pre-esterification mixture that includes about 15 weight percent (“wt %”) to about 25 wt % of a fatty acid dimer, based on total weight of the pre-esterification mixture, about 15 wt % to about 25 wt % of glycerin, based on the total weight of the pre-esterification mixture, and about 55 wt % to about 70 wt % of a hydrogenated natural oil, based on total weight of the pre-esterification mixture. The natural based-petrolatum composition, as described herein in any embodiment, may include one or more of the following: i) an acid value of less than about 5.0; ii) a polydispersity index of greater than about 1.3; or an iodine value of less than about 10.0.

The present disclosure provides a natural oil-based petrolatum composition that includes an esterification product of a pre-esterification mixture that includes about 15 wt % to about 25 wt % of a fatty acid dimer, based on total weight of the pre-esterification mixture, about 15 wt % to about 25 wt % glycerin, based on total weight of the pre-esterification mixture, and about 55 wt % to about 70 wt % of a hydrogenated natural oil, based on total weight of the pre-esterification mixture. The natural oil-based petrolatum composition, as described herein in any embodiment, may have one or more of the following: i) an acid value of less than about 1.5; ii) a polydispersity index of greater than about 1.3; or an iodine value of less than about 10.0.

The present disclosure further provides a method of making a natural oil-based petrolatum composition. The method includes mixing a hydrogenated natural oil, a fatty acid dimer, and glycerin to form a pre-esterification mixture; and adding a caustic or enzymatic catalyst to the mixture to facilitate a transesterification reaction until the mixture achieves an acid value (AV) of less than about 5.0, so as to obtain a natural oil-based petrolatum composition.

The present disclosure further provides a method of making a natural oil-based petrolatum composition. The method includes mixing a hydrogenated natural oil, a fatty acid dimer, and glycerin to form a pre-esterification mixture; and adding a caustic or enzymatic catalyst to the mixture to facilitate a transesterification reaction until the mixture has one or more of the following: i) an acid value of less than about 1.5; ii) a polydispersity index of greater than about 1.3; or an iodine value of less than about 10.0.

The natural oil-based petrolatum compositions described herein are useful within personal care or cosmetic products. In the case of personal care products specifically, it is desirable for the petrolatum substitute (i.e., natural oil-based petrolatum) to have properties which can improve ease of manufacturing while providing a pleasing appearance and feel.

Advantages, some of which are unexpected, are achieved by embodiments of the present disclosure. For example, various compositions described herein advantageously spread evenly and uniformly on the skin. They have a much more consistent rheology over a range of temperatures and more closely mimic the characteristics of petroleum-based petrolatum. The natural oil-based petrolatum compositions disclosed herein have

The natural oil-based petrolatum composition of the present disclosure also has improved manufacturing properties.

As a further advantage, various compositions described herein are natural oil-based and thus have the advantage of comprising biodegradable, renewable, and environmentally-friendly components. For example, the natural oil-based petrolatum composition of the present disclosure can be prepared from natural oils and yet can offer the above-described advantages.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter. One aspect described in conjunction with a particular embodiment is not necessarily limited to that embodiment and can be practiced with any other embodiment(s).

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, the singular forms “a,” “an,” and “the” and similar referents in the context of describing the elements (especially in the context of the following claims) include plural referents unless the context clearly dictates otherwise. For example, reference to “a substituent” encompasses a single substituent as well as two or more substituents, and the like. It is understood that any term in the singular may include its plural counterpart and vice versa, unless otherwise indicated herein or clearly contradicted by context.

The term “or” is used to refer to a nonexclusive “or” unless otherwise indicated. The statement “at least one of A and B” has the same meaning as “A, B, or A and B.”

In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Any publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the terms “for example,” “for instance,” “such as,” or “including” are meant to introduce examples that further clarify more general subject matter. Unless otherwise specified, these examples are provided only as an aid for understanding the applications illustrated in the present disclosure, and are not meant to be limiting in any fashion.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, plus or minus within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

As used herein, the following terms have the following meanings unless expressly stated to the contrary.

As used herein, the term “natural oil” may refer to oil derived from plants or animal sources. The term “natural oil” includes natural oil derivatives, unless otherwise indicated. Examples of natural oils include, but are not limited to, vegetable oils, algae oils, animal fats, tall oils, derivatives of these oils, combinations of any of these oils, and the like. Representative non-limiting examples of vegetable oils include canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, camelina oil, pennycress oil, hemp oil, algal oil, jojoba oil, and castor oil. Representative non-limiting examples of animal fats include lard, tallow, poultry fat, yellow grease, and fish oil. Tall oils are by-products of wood pulp manufacture. In some aspects, the natural oil may be refined, bleached, and/or deodorized. In some aspects, the natural oil is present individually or as mixtures thereof.

As used herein, the term “hydrogenated natural oil” refers to partial, complete, or substantially complete hydrogenation of a natural oil. Partial or substantially complete hydrogenation of natural oils is well known in the art and many hydrogenated natural oils may be purchased on the market and are available from a variety of commercial sources.

As used herein, a “natural oil-based” composition means that the composition contains oils and fatty acids which are predominantly, substantially or entirely, derived from natural oils and natural oil derivatives. The natural oil-based composition may, in various embodiments, contain oils which are at least 50%, 55%, 60%, 65%, 70%, 75%, 80%, 85%, 90%, 95%, 99%, 99.9%, 99.99% or about 100% natural oil or hydrogenated natural oil.

A “monoacylglyceride” refers to a molecule having a glycerol moiety with a single fatty acid residue that is linked via an ester bond. The terms “monoacylglycerol,” “monoacylglyceride,” “monoglyceride,” and “MAG” are used interchangeably herein. Monoacylglycerides include 2-acylglycerides and 1-acylglycerides.

A “diacylglyceride” refers to a molecule having a glycerol moiety having two fatty acid residues linked via ester bonds. The terms “diacylglycerol,” “diacylglyceride,” “diglyceride,” and “DAG” are used interchangeably herein. Diacylglycerides include 1,2-diacylglycerides and 1,3-diacylglycerides.

A “triacylglyceride” refers to a molecule having a glycerol moiety that is linked to three fatty acid residues via ester bonds. The terms “triacylglycerol,” “triacylglyceride,” “triglyceride,” and “TAG” are used interchangeably herein.

The term “fatty acid” as used herein can refer to a molecule comprising a hydrocarbon chain and a terminal carboxylic acid group. As used herein, the carboxylic acid group of the fatty acid may be modified or esterified, for example as occurs when the fatty acid is incorporated into a glyceride or another molecule (e.g., COOR, where R refers to, for example, a carbon atom). Alternatively, the carboxylic acid group may be in the free fatty acid or salt form (i.e., COO″ or COOH). The ‘tail’ or hydrocarbon chain of a fatty acid may also be referred to as a fatty acid chain, fatty acid sidechain, or fatty chain. The hydrocarbon chain of a fatty acid will typically be a saturated or unsaturated aliphatic group. A fatty acid having N number of carbons, will typically have a fatty acid side chain having N−1 carbons. However, the subject application also relates to modified forms of fatty acids, e,g., dimerized fatty acids, and thus the term fatty acid may be used in a context in which the fatty acid has been substituted or otherwise modified as described. For example, in various embodiments, a fatty acid may be dimerized with another fatty acid to result in a dimerized fatty acid or fatty acid dimer. Unless otherwise specified, the term fatty acid as used herein refers to a non-dimerized fatty acid, while the term dimerized fatty acid and the like refer to the dimer forms of fatty acids.

An “acylglyceride” refers to a molecule having at least one glycerol moiety with at least one fatty acid residue that is linked via an ester bond. For example, acylglycerides can include monoacylglycerides, diacylglycerides, triacylglycerides and acylglyceride polymers. The group acylglycerides can be further refined by additional descriptive terms and can be modified to expressly exclude or include certain subsets of acylglycerides. For example, the phrase mono- and di-acylglycerides refers to MAGs (monoacylglycerides) and DAGs (diacylglycerides), while the phrase non-MAG/non-DAG acylglycerides refers to a group of acylglycerides which exclude MAGs and DAGs. As another example, acylglycerides comprising a C36 dimeric fatty acid residue refers only to those acylglycerides having the specified residue.

A “fatty acid residue” is a fatty acid in its acyl or esterified form.

The levels of particular types of fatty acids may be provided herein in percentages out of the total fatty acid content of an oil. Unless specifically noted otherwise, such percentages are weight percentages based on the total fatty acids, including free fatty acids and esterified fatty acids as calculated experimentally.

A “saturated” fatty acid is a fatty acid that does not contain any carbon-carbon double bonds in the hydrocarbon chain. An “unsaturated” fatty acid contains one or more carbon-carbon double bonds. A “polyunsaturated” fatty acid contains more than one such carbon-carbon double bond while a “monounsaturated” fatty acid contains only one carbon-carbon double bond. Carbon-carbon double bonds may be in one of two stereoconfigurations denoted cis and trans. Naturally-occurring unsaturated fatty acids are generally in the “cis” form.

Non-limiting examples of fatty acids include C8, C10, C12, C14, C16 (e.g., C16:0, C16:1), C18 (e.g., C18:0, C18:1, C18:2, C18:3, C18:4), C20 and C22 fatty acids. For example, the fatty acids can be caprylic (8:0), capric (10:0), lauric (12:0), myristic (14:0), palmitic (16:0), stearic (18:0), oleic (18:1), linoleic (18:2) and linolenic (18:3) acids.

The fatty acid composition of an oil can be determined by methods well known in the art. The American Oil Chemist's Society (AOCS) maintains analytical methods for a wide variety of tests performed on vegetable oils. Hydrolysis of the oil's components to produce free fatty acids, conversion of the free fatty acids to methyl esters, and analysis by gas-liquid chromatography (GLC) is the universally accepted standard method to determine the fatty acid composition of an oil sample. The AOCS Procedure Ce 1-62 describes the procedure used.

The terms “esterification” or “esterified” means the creation of an ester bond including: 1) the dehydration reaction of an alcohol with an acid; 2) transesterification, the reaction of an alcohol with an ester to form a new ester; or 3) interesterification, the rearrangement of fatty acids within an triacylglycerol structure.

The terms “fatty acid dimer” and “dimerized fatty acid” are interchangeably used herein and refer generally to a compound containing two fatty acid subunits in which the respective fatty acid side chains are covalently bound to each other, e.g., via a bond or a linking group. Thus, as described herein, the fatty acid dimer is a covalent fatty dimer. The fatty acid dimer can be a heterodimer or a homodimer. As used herein, the carboxylic acid group of the fatty acid dimer may be modified or esterified, for example as occurs when the fatty acid dimer is incorporated into a glyceride or is attached to another molecule. Suitable fatty acid dimers are commercially available, for example, Radiacid 0960 Hydrogenated Standard Dimer and Radiacid 0970 Distilled Dimer Acid (Oleon N.V., Belgium) and UNIDYME 18 Dimer Acid (Kraton Corporation, Houston, TX).

As an example, the dimerized fatty acid residue can have the structure:

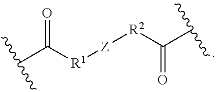

In the example dimerized fatty acid residue, R¹ and R² are each independently a substituted or unsubstituted aliphatic group. The aliphatic group can correspond to a saturated fatty acid side chain or an unsaturated fatty acid side chain having one, two, three or more double bonds. The aliphatic group can be, for example, 5 to 25 carbons, 7 to 21 carbons, 12 to 21 carbons, 15 to 19 carbons, or 17 carbons. Optionally, R¹ and R² can be substituted and example substituents include alkyl, alcohol, halide, and oxygen so as to form an epoxide ring. R¹ and R² can be a saturated or unsaturated linear aliphatic group having 7, 9, 11, 13, 15, 17, 19 or 21 carbons. When R¹ and R² are each a 17-carbon saturated or unsaturated group, the resulting dimerized fatty acid residue has 36 carbons. R¹ and R² can comprise hydrogen, carbon, oxygen, and nitrogen atoms; or R¹ and R² can consist of carbon, hydrogen, and oxygen atoms; or R¹ and R² can consist of carbon and hydrogen atoms.

The linking group Z is a bond, an oxygen atom, or any other suitable linking group. The linking group Z may be attached to R¹ and R² via any position. For example, the linking group Z may be attached to a position at R¹ and R² other than the terminal carbons. As another example, R¹ and R² can be a linear aliphatic group which corresponds to a fatty acid side chain, and the linking group Z can be attached at omega number 2, 3, 4, 5, 6, 7, 8, 9, 10, 11, 12, 13, 14, 15, 16, 17, etc., or, alternatively the linking group Z can be linked at the terminal (ω-1) carbon. In another example, the Z group represents multiple bonds such that R¹ and R² are linked so as to form a carbocyclic or heterocyclic ring between them. When Z is a bond, the dimerized fatty acid residue may have the structure:

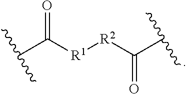

A “plurality” refers to two or more. For example, a polymeric compound having a plurality of glycerol units can have 2 or more glycerol units, 10 or more glycerol units, 100 or more glycerol units, 1,000 or more glycerol units, etc.

A “drop point” or “dropping point” generally refers to the temperature at which a material (such as a wax) softens and becomes sufficiently fluid to flow as determined under the conditions of a given standardized test. As used herein, drop points are determined via AOCS Standard Procedure Cc 18-80. (Official Methods and Recommended Practices of the American Oil Chemists' Society, 7th Edition). Drop point is similar to melting point in that it reflects the thermal characteristics of a compound, however, drop point can be useful in defining materials which do not have a defined melting point.

The term “Polydispersity Index” (also known as “Molecular Weight Distribution”) as used herein is the ratio of weight average molecular weight (Mw) to number average molecular weight (Mn). The polydispersity data is collected using a Gel Permeation Chromatography instrument equipped with a Waters 510 pump and a 410 differential refractometer. Samples are prepared at an approximate 2% concentration in a THF solvent. A flow rate of 1 ml/minute and a temperature of 35° C. are used. The columns consist of a Phenogel 5 micron linear/mixed Guard column, and 300×7.8 mm Phenogel 5 micron columns (styrene-divinylbenzene copolymer) at 50, 100, 1000, and 10000 Angstroms. Molecular weights were determined using the following standards:

| Standard | Mono-olein | Diolein | Arcol LHT 240 | Trio-lein |
|---|---|---|---|---|
| Mol. | 356 | 620 | 707 | 878 |
| Weight | | | | |
| (Daltons) | | | | |
| | Epoxidized | Acclaim | Mult-ranol | Acclaim |
| Standard | Soybean Oil | 2200 | 3400 | 8200 |
| Mol. | 950 | 2000 | 3000 | 8000 |
| Weight | | | | |
| (Daltons) | | | | |

The term “weight average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using the test described herein or through size exclusion chromatography, light scattering, small angle neutron scattering, X-ray scattering, and sedimentation velocity.

The term “number average molecular weight” as used herein refers to Mn, which is equal to the total weight of the sample divided by the number of molecules in the sample. Mn, can be represented by the formula ΣMᵢnᵢ/nᵢ, where nᵢ is the number of molecules of molecular weight Mᵢ.

The term “Acid Value” (AV) as used herein is defined as the weight of KOH in mg needed to neutralize the organic acids present in 1 g of test sample and it is a measure of the free fatty acids present in the composition. AV can be determined by the AOCS Official Method Cd 3d-63. The acid value of the compositions described herein may be less than 10.0, or less than 5.0, or less than 4.0, or between 0.5 and 5.0, or between 0.5 and 4.0.

The term “Hydroxyl Value” as used herein is defined as the hydroxyl value, expressed in milligrams of potassium hydroxide and corresponds to the number of hydroxyl groups present in 1 g of a sample, is one of the traditional characteristics of oils and fats. Hydroxyl Value may be determined by AOCS Standard Method Cd 13-60.

The term “Iodine Value” (commonly abbreviated as IV) as used herein is the mass of iodine in grams that is consumed by 100 grams of a chemical substance. Iodine numbers are often used to determine the amount of unsaturation in fats, oils and waxes. In fatty acids, unsaturation occurs mainly as double bonds which are very reactive towards halogens, iodine in this case. Thus, the higher the iodine value, the more unsaturation is present in the sample. The Iodine Value of a material can be determined by the standard well-known Wijs method (A.O.C.S. Cd1-25).

### Natural Oil-Based Petrolatum Composition

The natural oil-based petrolatum composition described herein has a unique composition which provides a more consistent rheology over a variety of temperatures more closely mimicking petroleum-based petrolatum.

The compositions of the present invention includes an esterification product of a pre-esterification mixture that includes a fatty acid dimer, glycerin, and a hydrogenated natural oil.

Prior to esterification, the pre-esterification mixture may include the fatty acid dimer in an amount of about 15 wt % to about 25 wt %; the glycerin at an amount of about 15 wt % to about 25 wt % and the hydrogenated natural oil in an amount of about 55 wt % to about 70 wt %.

In some embodiments, prior to esterification, the fatty acid dimer may be present in the pre-esterification mixture at about 18 wt % to about 23 wt %. In some embodiments, prior to esterification, the fatty acid dimer may be present in the pre-esterification mixture at about 15 wt %, about 16 wt %, about 17 wt %, about 18 wt %, about 19 wt %, about 20 wt %, about 21 wt %, about 22 wt %, about 23 wt %, about 24 wt %, about 25 wt %, or any range including and/or in between any two of the preceding values.

In some embodiments, the fatty acid dimer is Radiacid 0960 Hydrogenated Standard Dimer and Radiacid 0970 Distilled Dimer Acid (Oleon N.V., Belgium) and UNIDYME 18 Dimer Acid (Kraton Corporation, Houston, TX).

In some embodiments, the fatty acid dimer may be Radiacid 0970.

In some embodiments, prior to esterification, the hydrogenated natural oil may be present in the pre-esterification mixture at about 55 wt % to about 70 wt %. In some embodiments, prior to esterification, the hydrogenated natural oil may be present in the pre-esterification mixture at about 55 wt %, about 60 wt %, about 65 wt %, about 70 wt %, or any range including and/or in between any two of the preceding values. In some embodiments, prior to esterification, the hydrogenated natural oil may be present in the pre-esterification mixture at about 55 wt % to about 65 wt %.

In some embodiments, the hydrogenated natural oil may be hydrogenated soy oil, palm oil, canola oil, castor oil, coconut oil, or combinations thereof. In some embodiments, the hydrogenated natural oil is hydrogenated soy oil.

In some embodiments, the hydrogenated natural oil is a blend of two or more hydrogenated natural oils selected from hydrogenated soy oil, hydrogenated palm oil, hydrogenated canola oil, hydrogenated castor oil, or hydrogenated coconut oil.

In some embodiments, prior to esterification, the glycerin may be present in the pre-esterification mixture in an amount of about 18 wt % to about 23 wt %. In some embodiment, prior to esterification, the glycerin may be present in the pre-esterification mixture in an amount of about 15 wt %, about 16 wt %, about 17 wt %, about 18 wt %, about 19 wt %, about 20 wt %, about 21 wt %, about 22 wt %, about 23 wt %, about 24 wt %, about 25 wt %, or any range including and/or in between any two of the preceding values.

Following esterification, the natural oil-based petrolatum composition may include minimal amounts of free fatty acids and glycerin. For example, the composition may include less than about 2 wt % free fatty acids and free glycerin. In another embodiment, the composition may include less than about 1 wt %, about 2.5 wt %, less than about 5 wt %, or less than about 10 wt %, free fatty acids, free glycerin and triacylglycerides.

In some embodiments, the acylglyceride polymer having at least two dimer structures is represented to by following: wherein R³ is hydrogen, glycerol, a substituted glycerol, or a fatty acid and n is one or greater.

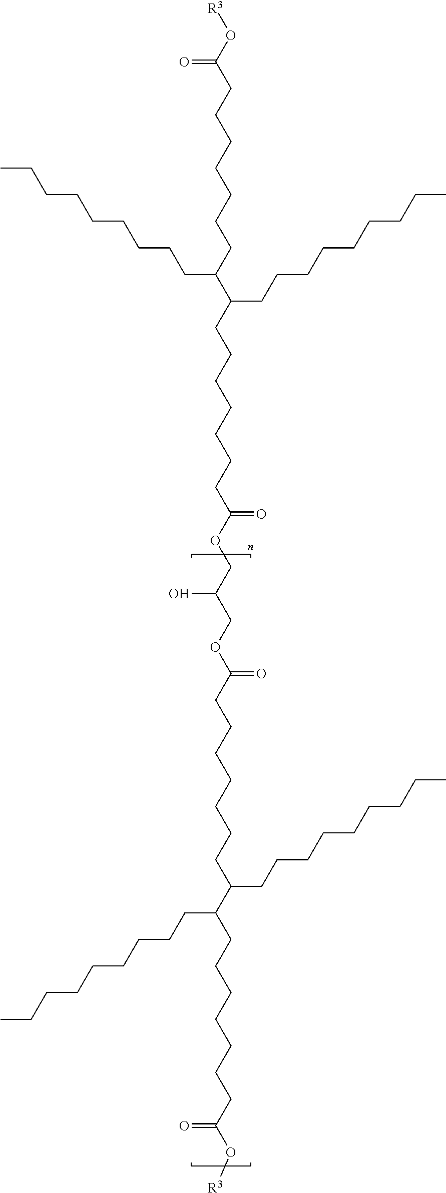

The composition may include about 5.0 wt. % to about 50 wt. % of acylglyceride polymers having at least two dimer structures. Alternatively, the composition may include greater than 10% or about 5.0 wt. % to about 50 wt. % of acylglyceride polymers having at least two dimer structures.

The natural oil-based petrolatum composition of the present invention can further be described in terms of average molecular weight distribution, which may be determined by gel permeation chromatography (GPC).

The natural based-petrolatum composition, as described herein in any embodiment, may include one or more of the following: i) an acid value of less than about 5.0; ii) a polydispersity index of greater than about 1.3; or an iodine value of less than about 10.0.

The acid value as described herein in any embodiment may be less than about 5.0 mg KOH/g, less than about 1.5 mg KOH/g, or about 0.1 mg KOH/g to about 1.5 mg KOH/g. For example, in any embodiment disclosed herein, the acid value may be about 0.1 mg KOH/g to less than about 5.0 mg KOH/g, about 0.1 mg KOH/g to about 3.5 mg KOH/g, about 0.1 mg KOH/g to about 1.5 mg KOH/g or any range including and/or between any two of the preceding values.

The iodine value of the compositions described herein may be less than about 10.0, or less than about 5.0, or in between about 0.5 to about 4.5. Suitable iodine values as described herein in any embodiment may include about 0.5, about 1.0, about 1.5, about 2.0, about 2.5, about 3.0, about 3.5, about 4.0, about 4.5, about 5.5, about 6.0, about 6.5, about 7.0, about 7.5, about 8.0, about 8.5, about 9.0, about 9.5, about 10.0, or any range including and/or in between any two of the preceding values. For example, the iodine value may be about 0.5 to about 5.0, about 0.5 to about 4.5, about 1.0 to about 4.5, or about 2.5 to about 4.5.

The polydispersity index (PDI) of the composition, as described herein in any embodiment, may be greater than about 1.3. For example, the composition may have a PDI of about 1.3 to about 2.0 or from about 1.3 to about 1.7.

The natural-based petrolatum compositions described herein can be a semisolid material that can hold its own shape but deflects under pressure similar to a grease or shortening. Resistance to deflection under pressure can be determined through use of a cone penetration test. Cone penetration can be measured by use of standard methodology ASTM D217-2.

The natural oil-based petrolatum exhibits a combination of rheological properties that provides for comparable spreading and tackiness to petroleum-based petrolatum. In any embodiment disclosed herein, the natural oil-based petrolatum exhibits one or more rheological properties selected from a drop point of about 30° C. to about 60° C., a cone penetration at 25° C. of greater than 20 or from about 20 to about 100 or from about 40 to about 60 (Dmm ( 1/10 of mm), kinetic viscosity at 100° C. of about 5 mm²/s to about 35 mm²/s, a congealing point of about 25° C. to about 45° C., or combinations thereof.

### Method of Preparing Natural Oil-Based Petrolatum Composition

The present disclosure also provides a method of making a natural oil-based petrolatum composition. The method involves mixing a hydrogenated natural oil, a fatty acid dimer, and glycerin. The resulting mixture is treated with an esterification catalyst which induces esterification and transesterification. The reaction is allowed to proceed until the reaction mixture reaches an acid value of less than 5.0 mg KOH/g or until the reaction mixtures reaches an acid value of less 1.5 mg KOH/g so as to provide a natural oil-based petrolatum composition. In some embodiments that reaction mixture reaches an acid value of about 0.1 mg KOH/g to about 1.5 mg KOH/g. In some embodiments that reaction mixture reaches an acid value between 0.5 mg KOH/g and 1.5 mg KOH/g.

The natural oil can be a vegetable oil or an animal oil. Examples of natural oils may include, but are not limited to, canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, camelina oil, pennycress oil, hemp oil, algal oil, castor oil, lard, tallow, poultry fat, yellow grease, fish oil, or mixtures thereof. The natural oil as described herein in any embodiment may be a hydrogenated natural oil.

In various embodiments, the fatty acid dimer has the structure

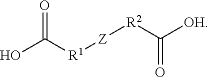

R¹ and R² are each independently defined divalent fatty acid chains so that R¹ and R² may be the same or different. When R¹ and R² are the same, the dimerized fatty acid represents a fatty acid homodimer. When R¹ and R² are the different, the dimerized fatty acid represents a fatty acid heterodimer. In various embodiments, each of R¹ and R² is independently a substituted or unsubstituted C₇-C₂₁ aliphatic group corresponding to a saturated chain or an unsaturated fatty acid side chain having one, two, three or more double bonds. R¹ and R² can represent substituted forms of the side chains of naturally occurring fatty acids. For example, R¹ and R² may each independent be a saturated or unsaturated linear aliphatic group having 7, 9, 11, 13, 15, 17, 19 or 21 carbons. When R¹ and R² are each a 17-carbon saturated or unsaturated group, the resulting dimerized fatty acid has 36 carbons. R¹ and R² can comprise hydrogen, carbon, oxygen, and nitrogen atoms; or R¹ and R² can consist of carbon, hydrogen, and oxygen atoms; or R¹ and R² can consist of carbon and hydrogen atoms.

The linking group Z is a bond, an oxygen atom, or a sulfur atom. The linking group Z may be attached to R¹ and R² via any position. When Z is a bond, the dimerized fatty acid may have the structure:

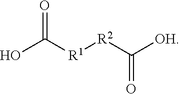

Non-limiting examples of dimerized fatty acids include those commercially available as Radiacid 0960 Hydrogenated Standard Dimer and Radiacid 0970 Distilled Dimer Acid (Oleon N.V., Belgium) and UNIDYME 18 Dimer Acid (Kraton Corporation, Houston, TX). The dimerized fatty acid may be derived from a natural oil. As another example, a T18 dimer acid can be used. Radiacid 0960 Distilled Dimer Acid (Oleon N.V., Belgium) as used herein was analyzed to contain 1.6% monomer, 79.22% dimer, 14.99% trimer, and 4.19% tetramer or higher.

The method described herein may comprise the following steps. A reaction mixture of hydrogenated natural oil(s), glycerin, and fatty acid dimer are pre-melted and heated to a temperature ranging from 60-80° C. before adding to a reaction vessel along with a nitrogen sparge to prevent oxidation.

The reaction mixture has the composition described herein and mixture is treated to induce chemical or enzymatic transesterification and esterification by methods well known in the art.

To carry out chemical transesterification, a catalyst can be added at an amount of about 0.1 wt % relative to the reaction mixture. Example catalysts can be potassium hydroxide or calcium hydroxide. The reaction temperature can then be increased to about 200-250° C. This reaction temperature is maintained until an acid value of less than 5.0 is achieved or a polydispersity index of greater than 1.3 is obtained. An acid, for example a mineral acid such as phosphoric acid, can be added at an amount of about 0.2 wt % to neutralize the catalyst with a slight excess. The reaction mixture can then be cooled to a temperature ranging from about 60-80° C. A filter media, for example acid activated beaching clay, can be added to the reaction mixture in an amount of about 2 wt % relative to the reaction mixture to remove impurities. The final product, i.e., the natural oil-based petrolatum composition, is then filtered to remove the salt and clay mixture.

Alternatively, to carry out enzymatic transesterification, an enzymatic catalyst can be added at an amount of 2 wt % relative to the reaction mixture. An example enzymatic catalyst can be Lipase Novozyme 435. A vacuum of about 50 torr can be used to remove water as the reaction is taking place. A reaction temperature ranging from about 60-80° C. is maintained until an acid value of less than 5.0 is achieved or a polydispersity index of greater than 1.3 is obtained. The enzymatic catalyst can then be filtered out using an appropriate filter device to obtain the final product, i.e., the natural oil-based petrolatum composition.

### Topical Formulation

The emulsion provided herein is useful in the manufacture of topical formulations such as personal care products or cosmetics. The inventors unexpectedly found that formulations comprising a natural oil-based petrolatum as described herein have numerous desirable characteristics as explained further below and can be used to replace all or part of the petroleum-based petrolatum currently used in personal care or cosmetic formulations.

In one aspect, the present invention is a topical formulation comprising a natural oil-based petrolatum as described herein. As used herein, the term “topical formulation” refers to a formulation that may be applied directly to a part of the body. The term “formulation” is used herein to denote compositions of various ingredients in various weight ranges, in accordance with the present disclosure for use in personal or home care.

“Personal care” means and comprises any cosmetic, hygienic, toiletry and topical care products including, without limitation, leave-on products (i.e., products that are left on the skin or keratinous substrates after application); rinse-off products (i.e., products that are washed or rinsed from the skin and keratinous substrates during or within a few minutes of application); shampoos; hair curling and hair straightening products; combing or detangling creams; hair style maintaining and hair conditioning products (either concentrated masks or more standard formulations; whether rinse-off or leave-on); lotions and creams for nails, hands, feet, face, scalp and/or body; hair dye; face and body makeup; foundation; masks; nail care products; astringents; deodorants; antiperspirants; anti-acne; antiaging; depilatories; colognes and perfumes; skin protective creams and lotions (such as sunscreens); skin and body cleansers/body washes; face cleansers; skin conditioners; skin toners; skin firming compositions; skin tanning and lightening compositions; liquid soaps; bar soaps; syndet bars; bath products; shaving products; personal lubricants, and oral hygiene products (such as toothpastes, oral suspensions, and mouth care products).

The natural oil-based petrolatums disclosed herein can be utilized alone on the skin or hair and are particularly useful in reducing or replacing the various components in shampoos, body washes, and conditioner formulations or any conditioning formulations.

The texture of such personal care formulations is not limited and may be, without limitation, a liquid, gel, spray, emulsion (such as lotions and creams), shampoo, conditioner, combing cream, pomade, foam, tablet, stick (such as lip care products), makeup, suppositories, among others, any of which can be applied to the skin or hair and which typically are designed to remain in contact therewith until removed, such as by rinsing with water or washing with shampoo or soap or syndet bars. Other forms could be gels that can be soft, stiff, or squeezable. Sprays can be non-pressurized aerosols delivered from manually pumped finger-actuated sprayers or can be pressurized aerosols such as mousse, spray, or foam forming formulation, where a chemical or gaseous propellant is used.

Formulations prepared using the natural oil-based petrolatum disclosed herein have a white or pale white color that is generally considered to be aesthetically appealing. In some cases, the formulations of this disclosure may be further processed to make a colored end product. In such cases, the white color is beneficial because it will show up the additional pigment without influencing the final color.

Formulations containing the natural oil-based petrolatum of the present disclosure may optionally contain additional ingredients to tailor the viscosity to the needs of the particular application. A skilled artisan will readily appreciate the range of additives available to suit this purpose including but not limited to the following: sclerotium gum, xanthan gum, carrageenan, gellan gum, native starches, modified starches, sodium starch octenyl succinate, aluminum starch succinate, hydroxypropyl starch phosphate, pectin, calcium citrate, salt(s) NaCl, KCl, acrylate polymers, acrylate based copolymers, carbomers, cellulose, citrus fibres and derivatives, hydroxy ethyl cellulose, carboxy methyl cellulose, polyols such as sorbitol, and mixtures thereof. These additives may be utilized to add texture, viscosity, or structure to the formulations. A skilled artisan would appreciate that they may be present in various concentrations depending on the needs of the particular formulation and may even be the predominant element of a particular formulation. Additional texturizers may, or may not be used, in formulations including the anhydride modified starches disclosed herein and will depend on the needs of the formulation and objective of the product being prepared. It may be desired to add additional texturizers to aid in viscosity when the anhydride modified starch disclosed herein are used in shampoos or in hair conditioning formulations.

Formulations containing the natural oil-based petrolatum of the present disclosure may optionally contain at least one further ingredient chosen from the group consisting of preservative, salt, vitamin, emulsifier, texturizer, nutrient, micronutrient, sugar, protein, polysaccharide, polyol, glucose, sucrose, glycerol, sorbitol, pH adjusters, emollients, dyes, pigments, skin actives, oils, hydrogenated oils, waxes, or silicones.

Formulations containing the natural oil-based petrolatum of the present disclosure may have a wide range of pH values. Aspects of this disclosure include formulations having pH between 3-11, or between 4-8, or between 4-7.

Formulations of the present disclosure can contain any useful amount of the natural oil-based petrolatum of the present disclosure. Formulations will preferably contain between 1-100%, 50-99%, 75-95%, 20-90%, 20-80%, 1-30%, 2-20%, or 1-15% by weight natural oil-based petrolatum in the final formulations.

In some embodiments the personal product comprising the natural oil-based petrolatum is a body wash, face wash, shampoo, conditioner, combing cream, leave-on conditioner, skin moisturizer, lip moisturizer, or cosmetic.

In another embodiment, the esterification products of a pre-esterification mixture comprising: about 15 wt % to about 25 wt % of a fatty acid dimer, about 15 wt % to about 25 wt % of glycerin, and about 55 wt % to about 70 wt % hydrogenated natural oil, wherein the esterification product has an Iodine Value of less than 50, or less than 25, or less than 10.

In another embodiment, the esterification products of a pre-esterification mixture comprising: about 15 wt % to about 25 wt % of a fatty acid dimer, about 15 wt % to about 25 wt % of glycerin, and about 55 wt % to about 70 wt % hydrogenated natural oil, wherein the esterification product has an Iodine Value of between 1 and 50, or between 1 and 25, or between 1 and 10.

In another embodiment, the esterification products of a pre-esterification mixture comprising: about 15 wt % to about 25 wt % of a fatty acid dimer, about 15 wt % to about 25 wt % of glycerin, and about 55 wt % to about 70 wt % hydrogenated natural oil, wherein the esterification product has a poly dispersity index greater than 1.3, or greater than 1.5 or between about 1.3 and about 2.0 or between about 1.3 and about 1.7.

In another embodiment, the esterification products of a pre-esterification mixture comprising: about 15 wt % to about 25 wt % of a fatty acid dimer, about 15 wt % to about 25 wt % of glycerol, and about 55 wt % to about 70 wt % hydrogenated natural oil, wherein the esterification product has an acid value of less than 5.0 mg KOH/g or between 0.1 mg KOH/g and 1.5 mg KOH/g or between 0.5 mg KOH/g and 1.5 mg KOH/g.

Any and every combination of two or more features disclosed herein for the natural oil-based petrolatums has been specifically contemplated and envisioned by the inventors. Therefore, the inventors have conceived of, and accordingly disclosed, every combination of single points and ranges disclosed for fatty acid dimer, hydrogenated natural oil, and glycerin ratios; as well as each and every combination of one or more of the value or ranges of the following parameters: drop melting point, cone penetration, kinetic viscosity, congealing point, hydroxyl value, acid value, iodine value, and polydispersity index.

### EXAMPLES

TABLE 1

| Materials | Source |
|---|---|
| Radiacid 0970 Distilled Dimer | Kraton Corporation, Houston, TX |
| Acid (Oleon N.V., Belgium | |
| Fully Hydrogenated Coconut Oil | Cargill Incorporated Regal 92 |
| Fully Hydrogenated Soybean Oil | Cargill Incorporated S-155 |
| Glycerol | Cargill Incorporated |

### Example 1

The following chemical transesterification method was carried out to make Sample C1. All components (including dimer) or oils as described in Table 2 were pre-melted and heated to 70° C. before adding to the reaction vessel under a nitrogen sparge to keep the product from oxidizing during the reaction. The agitator was turned on to mix the contents. A caustic catalyst was added (Potassium Hydroxide (KOH) or Calcium Hydroxide (Ca(OH)₂)) at 0.1% dosage. Once all ingredients were added and well mixed the temperature was increased to 200° C. to 250° C. The reaction temperature was maintained until an acid value of 10 or less was achieved. An acid, Phosphoric Acid (85% concentration), was added at 0.2% to neutralize the catalyst with a slight excess. The mixture was cooled to 70° C. and an acid activated bleach clay, B80, was added to the reaction at 2% and allowed to absorb the salts from the catalyst. The product was then filtered to remove the salts and clay mixture as well as other impurities.

TABLE 2
Examples

| Components by Weight | C1 |
|---|---|
| Fatty acid dimer (T18 Dimer) | 20% |
| Fully Hydro Coconut Oil | 15% |
| Fully Hydro Soy Oil | 45% |
| Glycerin | 20% |
| Drop melting Point (° C.) | 37.2 |
| Cone Penetration at 25° C. | 51 Dmm |
| Kinetic Viscosity at 100° C. (mm²/s) | 18.75 |
| Lovibond Color 5 1/4 Ly/Lr | 12.0/2.7 |
| Congealing point (° C.) | 28.5 |
| Hydroxyl Value | 304.4 |
| Acid value | 0.98 |
| Iodine Value | 4.5 |
| TGA | |
| Mn/Mw (Da) | 712/1020 |
| PDI | 1.433 |

1. A natural oil-based petrolatum composition comprising the esterification product of a pre-esterification mixture that includes:
about 15 wt % to about 25 wt % of fatty acid dimer, based on total weight of the pre-esterification mixture;
about 15 wt % to about 25 wt % of glycerin, based on total weight of the pre-esterification mixture; and
about 55 wt % to about 70 wt % of hydrogenated natural oil, based on total weight of the pre-esterification mixture;
wherein the natural oil-based petrolatum product has an acid value of less than about 5.0 mg KOH/g.

2. The natural oil-based petrolatum of claim 1, wherein the acid value is less than about 1.5 mg KOH/g.

3. The natural oil-based petrolatum of claim 1, wherein the acid value is about 0.1 mg KOH/g to about 1.5 mg KOH/g.

4. The natural oil-based petrolatum of claim 1, wherein the pre-esterification mixture comprises about 18 wt % to about 23 wt % of the fatty acid dimer.

5. The natural oil-based petrolatum of claim 1, wherein the pre-esterification mixture comprises about 18 wt % to about 23 wt % of the glycerin.

6. The natural oil-based petrolatum of claim 1, wherein the pre-esterification mixture comprises about 55 wt % to about 65 wt % of the hydrogenated natural oil.

7. The natural oil-based petrolatum of claim 1, wherein the hydrogenated natural oil comprises hydrogenated soy oil, hydrogenated palm oil, hydrogenated canola oil, hydrogenated castor oil, hydrogenated coconut oil, or combinations thereof.

8. The natural oil-based petrolatum of claim 7, wherein the hydrogenated natural oil comprises at least two or more hydrogenated soy oil, hydrogenated palm oil, hydrogenated canola oil, hydrogenated castor oil, hydrogenated coconut oil, or combinations thereof.

9. The natural oil-based petrolatum of claim 1, wherein the natural oil-based petrolatum further comprises an Iodine Value of less than about 10.0.

10. The natural oil-based petrolatum of claim 1, wherein the natural oil-based petrolatum further comprises a polydispersity index of greater than about 1.3.

11. The natural oil-based petrolatum of claim 1, wherein the natural oil-based petrolatum exhibits one or more rheological properties selected from a drop point of about 30° C. to about 60° C., a cone penetration at 25° C. from about 20 to about 100 (Dmm ( 1/10 of mm)), kinetic viscosity at 100° C. of about 5 mm²/s to about 35 mm²/s, a congealing point of about 25° C. to about 45° C., or combinations thereof.

12. A personal care product comprising a natural oil-based petrolatum composition wherein the natural oil-based petrolatum composition comprises the esterification product of a pre-esterification mixture that includes:
about 15 wt % to about 25 wt % of a fatty acid dimer, based on total weight of the pre-esterification mixture;
about 15 wt % to about 25 wt % of a glycerin, based on total weight of the pre-esterification mixture; and
about 55 wt % to about 70 wt % of a hydrogenated natural oil, based on total weight of the pre-esterification mixture;
wherein the natural oil-based petrolatum product has an acid value of less than about 5.0 mg KOH/g.

13. The personal care product of claim 12, wherein the natural oil-based petrolatum has acid value less than about 1.5 mg KOH/g.

14. The personal care product of claim 12 wherein the natural oil-based petrolatum has acid an acid value of about 0.1 mg KOH/g to about 1.5 mg KOH/g.

15. The personal care product of claim 12, which is a body wash, face wash, shampoo, conditioner, combing cream, skin moisturizer, skin lotion, lip moisturizer, or cosmetic.

16. A process of making the natural oil-based petrolatum composition, comprising:
mixing a hydrogenated natural oil, a fatty acid dimer, and glycerin to obtain a pre-esterification mixture;
treating the pre-esterification mixture with an esterification catalyst to obtain a reaction mixture; and
reacting the reaction mixture until the reaction mixture reaches an acid value of less than about 5.0 mg KOH/g to obtain the natural oil-based petrolatum composition.
